(19) United States
(12) Patent Application Publication
(10) Pub. No.: US 2025/0213479 A1
(43) Pub. Date: Jul. 3, 2025

(54) Ionic Liquid-Incorporated Lipid Nanoparticles

(71) Applicant: Duquesne University of the Holy Spirit, PITTSBURGH, PA (US)

(72) Inventors: Purva Prasad Khare, Pittsburgh, PA (US); Eden E.L. Tanner, Oxford, MA (US); Devika Soundara Manickam, Pittsburgh, PA (US)

(73) Assignee: Duquesne University of the Holy Spirit, PITTSBURGH, PA (US)

(21) Appl. No.: 18/971,892

(22) Filed: Dec. 6, 2024

Related U.S. Application Data
(60) Provisional application No. 63/606,869, filed on Dec. 6, 2023.

(51) Int. Cl. A61K 9/1272; A61K 9/00; A61K 9/51

(52) U.S. Cl. CPC A61K 9/1272; A61K 9/0019; A61K 9/5123

(57) ABSTRACT

The present invention is an improvement in Lipid Nanoparticles (LNPs) for re-routing their distribution from the liver and spleen (the non-target organs) to hard-to-deliver targets such as the brain and other tissues for drug delivery. Specifically, the invention derivatizes or otherwise modifies LNPs to provide the surface or constituency thereof with “Ionic Liquid” (IL) so that the IL-LNP incorporates an ionizable lipid such as a cationic ionizable lipid, an optional helper lipid, cholesterol and a (true) ionic liquid component. Among other benefits of the inclusion of “Ionic Liquid” is to decrease the delivery of LNPs to the body's filtering organ—the liver—and thereby re-route delivery to other target organs.

### CROSS-REFERENCE TO RELATED APPLICATION

This patent application claims priority to, and incorporates herein by reference, U.S. application Ser. No. 63/606,869 filed 6 Dec. 2023.

### BRIEF DESCRIPTION OF THE INVENTION

The present invention is an improvement in Lipid Nanoparticles (LNPs) for re-routing their distribution from the liver and spleen (the non-target organs) to the hard-to-deliver targets including the brain and other tissues for drug delivery. Specifically, the invention derivatizes or otherwise modifies LNPs to provide the surface or constituency thereof with “Ionic Liquid.” Among other benefits of the inclusion of “Ionic Liquid” is to impart the ability of the LNPs to cross the Blood-Brain Barrier (BBB). A non-limiting, exemplary way of providing Ionic Liquid is to replace the typical polyethyleneglycol dimyristoyl glycerol (PEG-DMG) component typically found in LNPs with (for example) choline trans-2-hexenoate ionic liquid. The ionic liquid functionality imparted by the exemplary choline trans-2-hexenoate ionic liquid (or other ionic liquid functionalities discussed herein) can significantly improve transfer of the LNPs to and across the BBB, as described further below.

### BRIEF DESCRIPTION OF THE FIGURES

FIG. 1 a)-c) are bar graphs illustrating particle sizes (a), dispersity indices (b), and zeta potential (c) of IL-LNPs measured using dynamic light scattering;

FIG. 2 includes four Transmission Electron Microscopy (TEM) images of standard LNPs (a) and IL-LNPs (b);

FIG. 3 is a bar graph which depicts the percentage of Red Blood Cells (RBCs) that hitchhiked LNPs for standard and IL-modified LNPs; and

FIG. 4 is a “heat map” of relative fluorescence intensities of the liver and spleen tissues from mice imaged using an IVIS Lumina XR imager.

### DETAILED DESCRIPTION OF THE INVENTION

The invention derivatizes or modifies Lipid Nanoparticles (LNPs) with an “Ionic Liquid,” such as choline trans-2-hexenoate ionic liquid, to impart the ability of the LNPs to cross the BBB. This derivatization or modification does not simply add ionic components to existing LNPs, however, because a typical PEG-DMG component of prior art LNPs is ordinarily avoided in the present invention, and therefore the broadest class of ionic liquid incorporated LNPs (IL-LNPs) according to the invention is made up of an ionizable lipid such as an ionizable cationic lipid, together with an optional helper lipid, cholesterol and an ionic liquid component. LNPs are FDA-approved drug carriers used (for example) in the Pfizer-BioNTech and Moderna COVID-19 mRNA vaccines. Ionic liquid-incorporated LNPs, such as those disclosed herein, can redirect nanoparticles to the brain tissue by decreasing drug uptake in the clearance organs such as the liver and spleen and also improving ability to cross the BBB. This new formulation is called IL-LNPs, which replace the polyethyleneglycol-dimyristoyl glycerol (PEG-DMG) in the standard LNP formulation with what are referred to her as ILs, or ionic liquids, even though as defined herein the IL contains more than just a simple ionic component alone. IL-LNPs are useful carriers to re-route the delivery of drugs to the brain tissue by decreasing the amount of drug delivered to clearance organs such as the liver and spleen. In particular, ILs have the ability to attach onto red blood cell surfaces that can facilitate the transport of IL-LNPs on red blood cells to distant organs such as the brain. Via this mechanism, LNP-ILs can more effectively reach the BBB via lower accumulation in the non-specific clearance organs such as the liver and spleen. Therefore, IL-LNPs serve as a platform technology to deliver small and large molecule drugs to the brain, a major challenge that is currently hindering effective treatment of brain diseases.

The single example of the addition of choline trans-2-hexenoate ionic liquid as the ionic-functionality imparting constituent is representative of a class of additives that generally include: an ionizable lipid such as an ionizable cationic lipid, an optional helper lipid, cholesterol and an additional ionic liquid. This additive generally replaces the PEG DMG component of LNP products that are, at this writing, already approved for delivery mRNA and siRNA active agents. By including this additive (replacing PEG DMG) the ingredients of the addition contribute to an emulsification effect in the LNP which, it is believed, align concentrically within the LNP to create ionic functionality, where previously the LNP was predominantly if not exclusively lipophilic.

The invention may be understood with particularity upon consultation to Example 1.

The feasibility of preparing and characterizing IL-LNPs is apparent from using two orthogonal techniques: dynamic light scattering and transmission electron microscopy. IL-incorporated LNPs have particle sizes and morphologies similar to a standard formulation of LNPs (that do not contain ILs). IL-LNPs are also well-tolerated by brain endothelial cells and motor neurons, suggestive of their safety for brain delivery and other biomedical applications. IL-LNPs also show superior uptake into brain endothelial cells and motor neurons when compared to the standard LNP formulation.

Extensive variation on the exemplary “ionic liquid” (IL) disclosed herein is possible, without departing from the scope of the present invention. The incorporation of cholesterol as a constituent is helpful for various reasons, including but not limited to its ability to emulsify with ionic components and, of course, its character as a “Generally Recognized as Safe” constituent in medicaments, vaccines, injectables, infusables and other pharmaceutical compositions. That the cationic lipid referred to is specifically cationic is more of a preferred embodiment than an inventive limitation—if an anionic lipid were to be substituted in the present invention, the inventive scope would still embrace such a variant. The “helper lipid” is described as optional, as is the second “ionic liquid,” because after one skilled in the art understands the present invention, widespread variation is possible within the high skill of the pharmaceutical engineering arts. It is not intuitive to consider making approved LNPs, known for their typical formulation including PEG-DMG, to create ionic functionality—in fact, the approved lipophilic LNPs themselves teach away from creating ionic functionality thereon. However, the present invention has identified the new and surprisingly improved results in formulating LNPs to impart ionic functionality thereto, with the benefits as described above and herein.

In light of the generalized disclosure above, the following provides additional alternatives and parameters by which to practice variants of the specific embodiments disclosed herein. The cholesterol component of the present invention can be cholesterol per se, or its analogs including but not limited to its analog naturally occurring substances Vitamin D2, Vitamin D3 or oleanolic acid. The optional helper lipids described herein can be DSPC (1,2-distearoyl-sn-glycero-3-phosphocholine), DOPE (1,2-dioleoyl-sn-glycero-3-phosphoethanolamine), DOTAP (1,2-dioleoyl-3-trimethylammonium propane) and DOPC (1,2-dioleoyl-sn-glycero-3-phosphocholine). The ionizable ionic lipid, such as an ionizable cationic lipid, can be replaced with variations of a lipid structure (6-((2-hexyldecanoyl)oxy)—N—(6-((2-hexyldecanoyl)oxy)hexyl)—N—(4-hydroxybutyl)hexan-1-aminium) with expected variations in the pKa of the amine, —OH or other groups, variations in the length of the carbon chains between the amine and the end group as well as variations in the length of the right side chain carbon tails. At the same time, a range of choline-trans-2-hexenoate ionic liquid with different cation:anion moral ratio (1:1, 1:2, 1:4 and 1:6) and newer IL compositions are within the skill of the art, once one knows to impart ionic capability to an LNP and to do so by incorporating ionizable lipids that will accommodate this innovation. Truly ionic components of the “Ionic Liquid” may be, for example, cations can include choline, quaternary ammonium compounds, and heterocyclic cations such as piperidinium, pyrrolidinium, imidazolium, morpholinium and pyridinium. Anions can be carboxylic acids with chains of 4-12 carbons, saturated, and with double bonds in the 2- and 3-positions and also branched varieties of the same. These compositions can be prepared with ratios of 1:1 through to 1:8 (cation:anion) by mole. Additional nonlimiting examples of quaternary ammonium compounds and carboxylic acids are listed as follows. Quaternary ammonium cations can include Methyltrimethylammonium, Ethyltrimethylammonium, Propyltrimethylammonium, Butyltrimethylammonium, Hexyltrimethylammonium, Octyltrimethylammonium, Decyltrimethylammonium, Dodecyltrimethylammonium, Tetradecyltrimethylammonium, Hexadecyltrimethylammonium, Benzyltrimethylammonium, Benzyltributylammonium, Benzyltriethylammonium, Benzalkonium cations, Dodecylbenzyldimethylammonium, Dimethyldioctylammonium, Dimethyldidecylammonium, Dimethyldidodecylammonium, Dimethyldihexadecylammonium, Tetraethylammonium, Tetrapropylammonium, Tetrabutylammonium, Tetramethylammonium, Triethylmethylammonium, Trimethylphenylammonium, Tetraphenylammonium, Methylpyridinium, Ethylpyridinium, Benzylpyridinium, Methylimidazolium, Ethylimidazolium, Cetylpyridinium, Quaternium-15, Stearyldimethylbenzylammonium, Laurylpyridinium, Didodecyldimethylammonium, Hexadecyldimethylbenzylammonium, Tetrabutylphosphonium-like ammonium variants, Alkyldimethylphenylammonium, Cetyltrimethylammonium, and Didecyldimethylammonium. Carboxylic acids can include Formic acid, Acetic acid, Propionic acid, Butyric acid, Isobutyric acid, Valeric acid, Isovaleric acid, Caproic acid, Enanthic acid, Caprylic acid, Pelargonic acid, Capric acid, Undecanoic acid, Lauric acid, Tridecanoic acid, Myristic acid, Pentadecanoic acid, Palmitic acid, Margaric acid, Stearic acid, Nonadecanoic acid, Arachidic acid, Behenic acid, Lignoceric acid, Cerotic acid, Oleic acid, Linoleic acid, Linolenic acid, Arachidonic acid, Palmitoleic acid, Myristoleic acid, Eicosenoic acid, Erucic acid, Crotonic acid, Tiglic acid, Acrylic acid, Methacrylic acid, 2-Methylbutyric acid, 3-Methylbutyric acid, 2-Ethylhexanoic acid, 3-Ethylpentanoic acid, 2,2-Dimethylpropanoic acid, 2,2-Dimethylbutanoic acid, and Dimethylbutanoic acid.

IL-LNPs are suitable for administration via parenteral routes commonly used for brain drug delivery, such as intrathecal, intracranial, or intravenous methods. The dosage is calculated based on the active ingredient, which in one instance is siRNA, and is adjusted according to the individual's body weight. For reference, the dosing regimen of Onpattro, a marketed siRNA therapeutic, is as follows: for patients weighing less than 100 kg, the recommended dose is 0.3 mg/kg administered once every three weeks. For patients weighing 100 kg or more, a fixed dose of 30 mg is administered every three weeks. We expect that a similar dosing strategy will be adapted to administering IL-LNPs.

The invention will be additionally illustrated by way of the following Example.

### Example 1

Lipid nanoparticles (LNPs) are FDA-approved drug carriers used in the Pfizer-BioNTech and Moderna COVID-19 mRNA vaccines. Ionic liquids (ILs) can potentially redirect nanoparticles to the brain or other hard-to-deliver tissues by decreasing drug uptake in the clearance organs such as the liver and spleen. We have created a new formulation called IL-LNPs by replacing the poly ethyleneglycol-dimyristoyl glycerol (PEG-DMG) in the standard LNP formulation using ILs. IL-LNPs are potentially promising carriers to re-route the delivery of drugs to the brain and other hard-to-deliver tissues by decreasing the amount of drug delivered to clearance organs such as the liver and spleen. We have also created IL-modified LNPs by coating LNP surfaces using ILs.

RESULTS The results of this Example, namely, particle size, dispersity index, and zeta potential of LNPs incorporating 1:1 and 1:2 choline trans-2-hexenoate ILs are shown in summary in Table 1 below.

TABLE 1
Representative formulation scheme for preparing IL-LNPs

| | | | |
|---|---|---|---|
| | | Stock | Volume of the |
| | w/w % in final | concentration | stock solution |
| Component | LNP mixture | (mg/mL) | required (μL) |

Ethanolic phase

| | | | |
|---|---|---|---|
| C12-200 | 50 | 1 | 12.5 |
| Cholesterol | 38.5 | 1 | 3.1 |
| DSPC | 10 | 0.1 | 16.9 |
| Ethanol | — | — | 92.5 |

| | |
|---|---|
| Total ethanolic phase volume | 125 |

Aqueous phase

| | | | |
|---|---|---|---|
| siRNA | 400 nM | 1 | 2.5 |
| 1:1 or 1:2 ILs | — | — | 12.5/25/50 |
| 10 mM citrate buffer | — | — | q.s. to 125 μL |

| | |
|---|---|
| Deionized water | 250 |
| Final volume of LNPs | 500 |
| prepared (μL) | |

IL-incorporated LNPs were prepared as described in Table 1 wherein the PEG-DMG component of standard LNPs was replaced by ILs. The ethanolic phase of LNPs was prepared with C12-200, cholesterol, and DSPC whereas the aqueous phase was prepared by adding 12.5, 25, or 50 μl of 1:1 or 1:2 (cation:anion) choline trans-2-hexenoate IL to the citrate buffer containing siRNA. Due to the hydrophilic nature of IL, they were incorporated into the aqueous phase instead of the organic phase. The lipid phase was added in a dropwise manner to the aqueous siRNA under continuous vortexing for 30 seconds using a Fisher benchtop vortexer at knob position ‘7’. A precalculated volume of 1× PBS pH 7.4 was added to the LNPs to adjust the final siRNA concentration to 400 nM.

IL-LNPs were prepared as described in Table 1 wherein the PEG-DMG component of LNPs was replaced by ILs. The ethanolic phase of LNPs was prepared with C12-200, cholesterol, and DSPC whereas the aqueous phase was prepared by adding either 12.5, 25, or 50 μL of the 1:1 or 1:2 choline trans-2-hexenoate ILs to the citrate buffer containing siRNA. Owing to the hydrophilic nature of the ILs, the ILs were incorporated into the aqueous phase instead of the organic phase. We noted precipitation of the citrate buffer and cloudiness of the aqueous phase upon the addition of 25 and 50 μL of the 1:2 ILs. Thus, we only prepared LNPs containing 12.5, 25, or 50 μL of the 1:1 ILs or 12.5 μL of the 1:2 ILs. PEG-DMG incorporated LNPs (No IL; with PEG) and LNPs without ILs and PEG-DMG (No IL; No PEG) were used as controls. We compared the stability of IL-LNPs in comparison to the standard LNPs prepared with the well-known steric stabilizer, PEG-DMG. The particle sizes, dispersity indices, and zeta potential of IL-LNPs were measured using Zetasizer Pro and compared to the controls 15-20 minutes post-preparation (day 0). We also tested the colloidal stability of IL-LNPs to study whether the LNPs maintain their sizes over a period of seven days upon storage at 2-8° C.

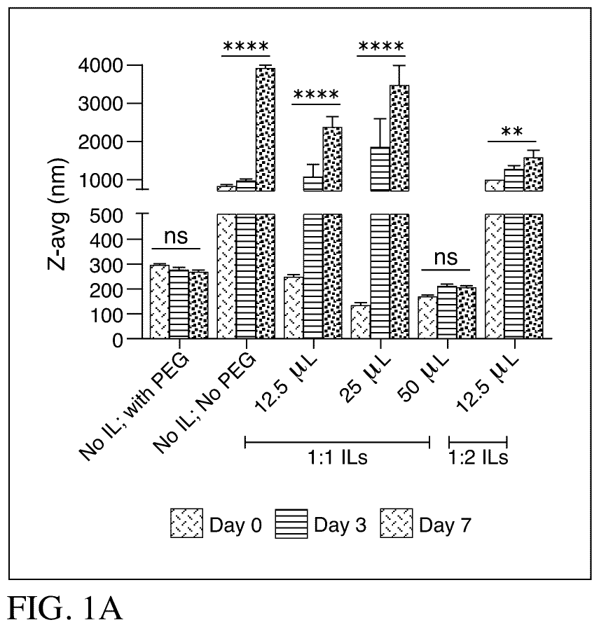

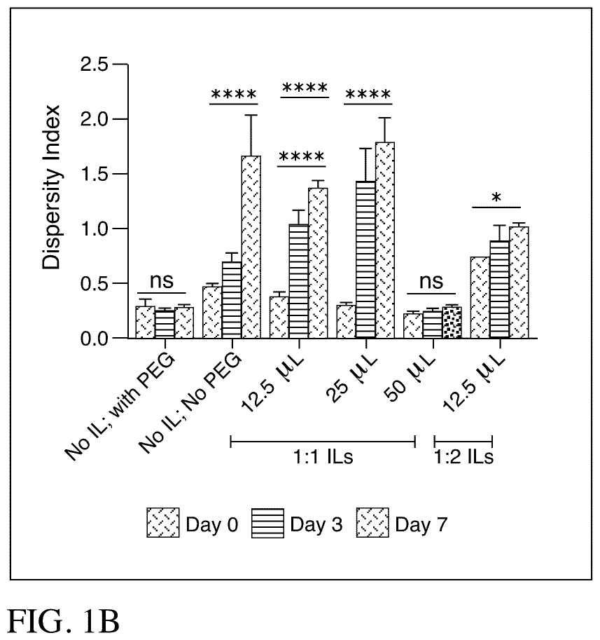

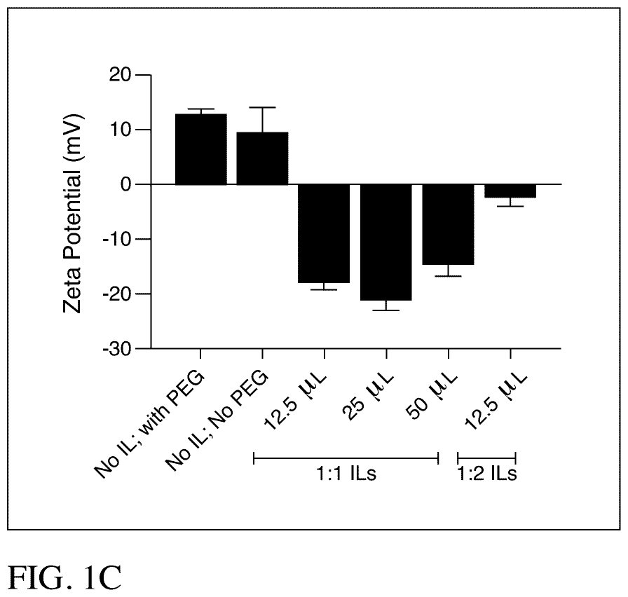

As may be seen in FIG. 1a)-c), particle sizes (a), dispersity indices (b), and zeta potential (c) of IL-LNPs were measured and reported using dynamic light scattering, for “No IL;” with “PEG or No IL;” “No PEG or I.: LNPs containing 400 nM siRNA were prepared in DI water and further diluted 10× using DI water. 12.5, 25 and 50 μL of 1:1 and 12.5 μL of 1:2 choline trans-2-hexenoate ILs were incorporated in the LNPs. Z-average particle diameters and dispersity indices were measured post-preparation and on days 3 and 7 and zeta potential was measured post-preparation (day 0) on a Malvern Zetasizer Pro. Statistical analysis was done using two-way ANOVA with Dunnett's multiple comparisons test. Data are presented as mean±SD of n=3 measurements. *p<0.05, **p<0.01, ****p<0.0001 and ns:nonsignificant.

As further results of the Example, we noted the following. LNPs prepared without PEG-DMG and ILs had significantly high diameters (˜ μm) and dispersity indices (˜0.45) that increased significantly after seven days. LNPs prepared with PEG-DMG had sizes of ˜270 nm that were maintained over a period of seven days. The dispersity index of LNPs prepared with PEG-DMG was ˜0.28 with similar values seven days post-preparation. LNPs prepared with the incorporation of 12.5 and 25 μL 1:1 ILs had sizes of ˜248 nm and ˜130 nm respectively denoting the stabilizing effect of the higher volume of ILs. However, sizes increased significantly post-seven-day storage exhibiting that these volumes of ILs were unable to stabilize the LNPs upon storage. Dispersity indices showed a similar trend wherein the values post-preparation were ˜0.3, increasing significantly upon storage. LNPs prepared with the incorporation of 12.5 μL 1:2 ILs had sizes and dispersity index of 980 nm and 0.74 which increased significantly after a seven-day storage regime. LNPs prepared with the incorporation of 50 μL 1:1 ILs had sizes and dispersity indices of ˜170 nm and ˜0.23 that were maintained over a period of seven days. Thus, a higher volume of ILs is necessary to get incorporated within the LNPs and stabilize them. Zeta potential measurements were performed to determine the change in the surface charge upon IL incorporation. IL-LNPs showed a shift in the zeta potential (negative values) as compared to LNPs without ILs (positive values). Overall, these results demonstrate that ILs can be incorporated into LNPs and IL-LNPs can maintain their colloidal properties for seven days.

For morphology of 1:1 choline trans-2-hexenoate IL-LNPs using Transmission Electron Microscopy in comparison to the standard LNPs, please see the below.

TABLE 2
Representative formulation scheme for preparing standard LNPs

| | | | |
|---|---|---|---|
| | | Stock | Volume of the |
| | w/w % in final | concentration | stock solution |
| Component | LNP mixture | (mg/mL) | required (μL) |

Ethanolic phase

| | | | |
|---|---|---|---|
| C12-200 | 50 | 1 | 12.5 |
| Cholesterol | 38.5 | 1 | 3.1 |
| PEG-DMG | 1.5 | 0.1 | 13.6 |
| DSPC | 10 | 0.1 | 16.9 |
| Ethanol | — | — | 78.9 |

| | |
|---|---|
| Total ethanolic phase volume | 125 |

Aqueous phase

| | | | |
|---|---|---|---|
| siRNA in 10 mM | 400 nM | 1 | 2.5 |
| citrate buffer | | | |

| | |
|---|---|
| Total aqueous phase volume | 125 |
| Final volume of LNPs | 500 |
| prepared (μL) | |

Overall, IL-LNPs showed sizes similar to standard LNPs (containing PEG-DMG). This observation was also noted in the DLS results. The shape and morphology of IL-LNPs were similar to standard LNPs. These results indicate that ILs can replace PEG-DMG to form stable nanoparticles. Referring now to FIG. 2, Transmission Electron Microscopy (TEM) images of standard LNPs (a) and IL-LNPs (b) were acquired using a Thermofisher T12 electron microscope.

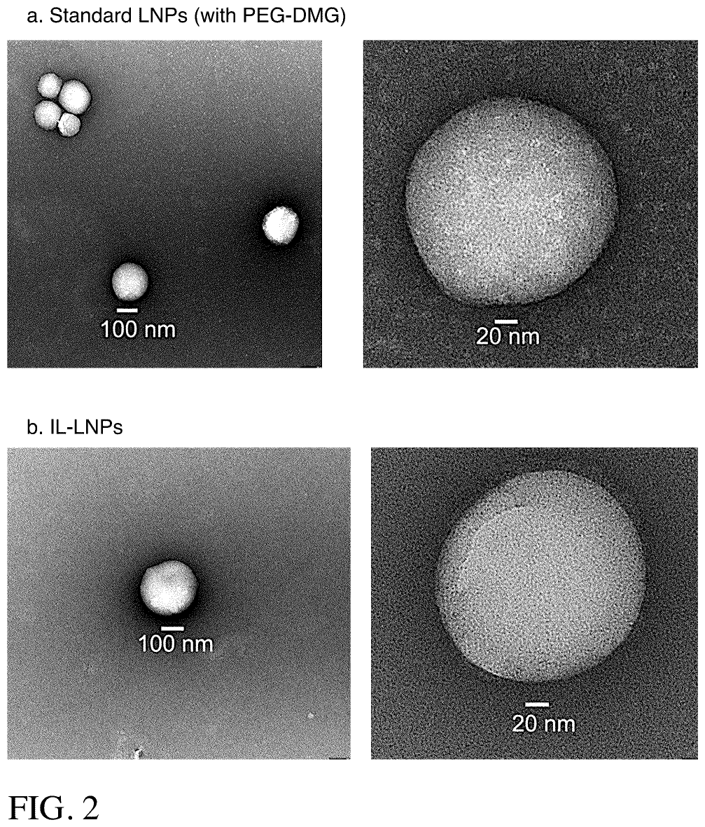

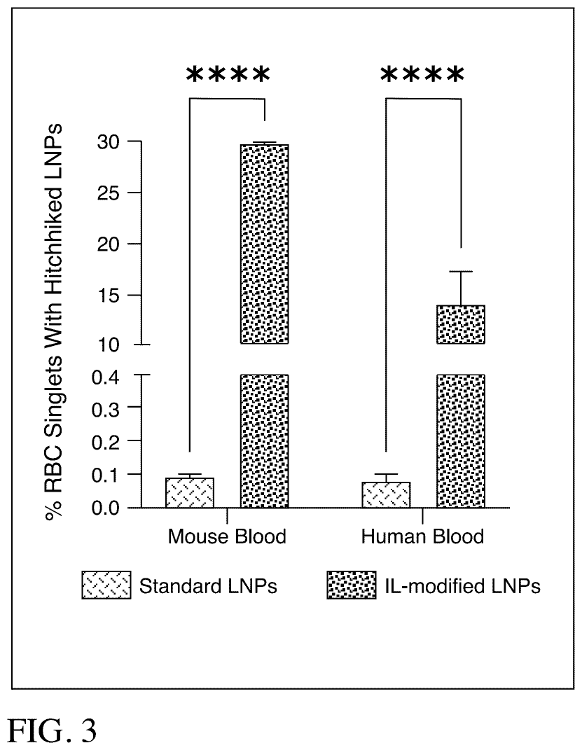

Further corroboration was determined as follows. Red blood cell (RBC) hitchhiking is a process by which nanoparticles associated with ILs attach to RBC membranes, facilitating their transport to target organs other than the liver—which is the natural homing site/organ for the standard LNPs. We evaluated the RBC hitchhiking ability of LNPs modified with 1:4 choline trans-2-hexenoate IL in mouse and human blood. As shown in FIG. 3, the percentage of 1:4 IL-modified LNPs hitchhiking on RBCs was dramatically greater than that of standard LNPs in both species. Additionally, the percentage of RBCs that hitchhiked LNPs increased by 300-fold in mouse blood and 150-fold in human blood, highlighting the superior hitchhiking ability of IL-modified LNPs compared to standard LNPs. These results point to the IL-modified LNP's rerouting drug delivery from the liver to difficult-to-deliver organs such as the brain. In particular, FIG. 3 shows the percentage of RBCs “that hitchhiked” LNPs for standard and IL-modified LNPs. Hitchhiking percentages were determined using flow cytometry, where Cy5-siRNA-loaded LNPs were mixed with whole blood (1:3 v/v RBC: LNP), incubated for 30minutes, and RBC singlets with LNPs were quantified. Data are presented as mean±SD. Statistical significance was assessed using one-way ANOVA with Dunnett's test (****p<0.0001).

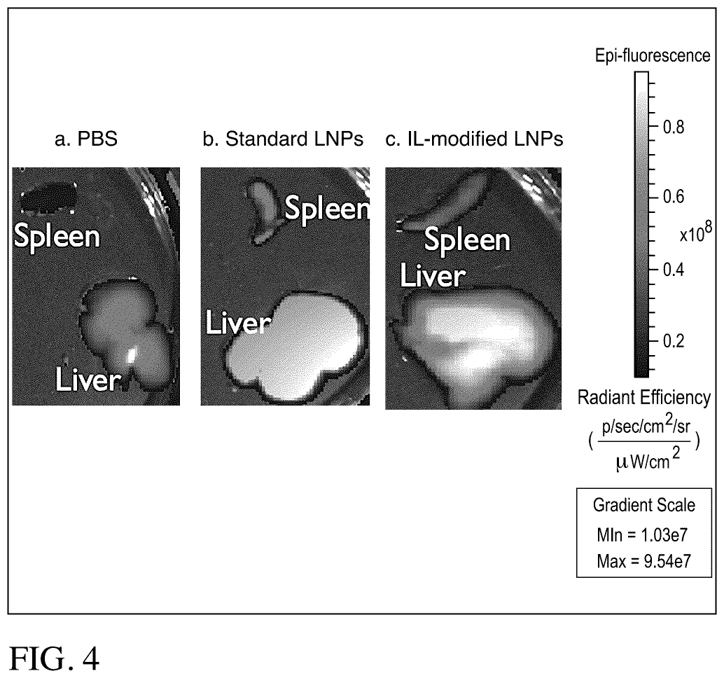

In vivo biodistribution of IL-modified LNPs in mice Based on the promising hitchhiking results presented above, ILs can modulate the biodistribution of LNPs, reducing their liver accumulation. To investigate this, we first prepared LNPs labeled with a lipophilic carbocyanine dye DiD: 1,1′-dioctadecyl-3,3,3′,3′-tetramethylindodicarbocyanine, 4-chlorobenzenesulfonate salt. DiD-labeled standard or IL-modified LNPs were injected into mice via retroorbital injection, with 1×PBS-injected mice as controls. Mice were euthanized, organs were isolated, washed with 1×PBS, and imaged using an IVIS Lumina XR imager (Caliper Lifesciences). As shown in FIG. 4, liver fluorescence intensity was lower in mice injected with IL-modified LNPs compared to standard LNPs, demonstrating the ability of IL-modified LNPs to decrease the liver accumulation of injected LNPs and re-route particle distribution to other organs.

Although the invention has been described with particularity above, with reference to particular constituents, method steps and amounts, the invention is only to be limited insofar as is set forth in the accompanying claims.

We claim:

1. An ionic liquid in combination with a lipid nanoparticle, wherein the present of the ionic liquid changes the lipophilic character of the lipid nanoparticle to impart a cationic or anionic charge thereto or therein.

2. The combination according to claim 1, wherein said ionic liquid is on the surface of said lipid nanoparticle.

3. The combination according to claim 1, wherein said ionic liquid is incorporated within said lipid nanoparticle.n

4. The combination according to claim 1, wherein an ionic liquid incorporated lipid nanoparticle includes an ionizable cationic lipid, an optional helper lipid, cholesterol and an ionic liquid.

5. The combination according to claim 1, wherein said combination is effective for delivery of drugs to reduce the hepatic first pass effect after administration.

6. The combination according to claim 1, wherein said combination is used as a carrier for at least one active agent or injectable for parenteral administration.

7. The combination according to claim 1, wherein said combination is effective to bind to red blood cells and potentially re-route LNP delivery or organs other than the liver by decreasing LNP delivery to the liver.
